(19) United States
(12) Patent Application Publication
(10) Pub. No.: US 2023/0019636 A1
(43) Pub. Date: Jan. 19, 2023

(54) INTERVERTEBRAL CAGE WITH NON-PARALLEL UNDERCUTS

(71) Applicant: CTL Medical Corporation, Addison, TX (US)

(72) Inventors: Jon SUH, Ambler, PA (US); Sean SUH, Milltown, NJ (US)

(21) Appl. No.: 17/947,926

(22) Filed: Sep. 19, 2022

Related U.S. Application Data
(63) Continuation of application No. 16/505,096, filed on Jul. 8, 2019.
Application No. 16/505,096 is a continuation-in-part of application No. 15/220,090, filed on Jul. 26, 2016, now abandoned.

(51) Int. Cl. A61F 2/44

(52) U.S. Cl. CPC A61F 2/447; A61F 2/442; A61F 2002/30784

(57) ABSTRACT

An intervertebral cage structure that comprises a main body having a first surface and a second surface located opposite to the first surface, a first plate disposed on the first surface of the main body, a second plate disposed on the second surface of the main body, and an opening formed at a center portion of the intervertebral cage structure and extending from the first plate to the second plate via the main body, wherein at least one of the first and second plates comprise non-parallel undercut portions.

### CROSS-REFERENCE TO RELATED APPLICATIONS

This application is a continuation of U.S. patent application Ser. No. 16/505,096 entitled “ACIF CAGE, CAGE SYSTEM AND METHOD,” filed Jul. 8, 2019, which is a continuation-in-part of U.S. patent application Ser. No. 15/220,090 entitled “ACIF CAGE, CAGE SYSTEM AND METHOD,” filed Jul. 26, 2016, the disclosures of which are incorporated by reference herein in their entireties.

### FIELD OF THE DISCLOSURE

The present disclosure relates generally to medical devices, and more specifically it relates to intervertebral and intradiscal devices, systems, and methods for deployment within a body of a patient.

### BACKGROUND OF THE DISCLOSURE

In mammals, the spinal (or vertebral) column is one of the most important parts. The spinal column provides the main support necessary for mammals to stand, bend, and twist.

In humans, the spinal column is generally formed by individual interlocking vertebrae, which are classified into five segments, including (from head to tail) a cervical segment (vertebrae C1-C7), a thoracic segment (vertebrae T1-T12), a lumbar segment (vertebrae L1-L5), a sacrum segment (vertebrae S1-S5), and coccyx segment (vertebrate Co1-Co5). The cervical segment forms the neck, supports the head and neck, and allows for nodding, shaking and other movements of the head. The thoracic segment attaches to ribs to form the ribcage. The lumbar segment carries most of the weight of the upper body and provides a stable center of gravity during movement. The sacrum and coccyx make up the back walls of the pelvis.

Intervertebral discs are located between each of the movable vertebra. Each intervertebral disc typically includes a thick outer layer called the disc annulus, which includes a crisscrossing fibrous structure, and a disc nucleus, which is a soft gel-like structure located at the center of the disc. The intervertebral discs function to absorb force and allow for pivotal movement of adjacent vertebra with respect to each other.

In the vertebral column, the vertebrae increase in size as they progress from the cervical segment to the sacrum segment, becoming smaller in the coccyx. At maturity, the five sacral vertebrae typically fuse into one large bone, the sacrum, with no intervertebral discs. The last three to five coccygeal vertebrae (typically four) form the coccyx (or tailbone). Like the sacrum, the coccyx does not have any intervertebral discs.

Each vertebra is an irregular bone that varies in size according to its placement in the spinal column, spinal loading, posture and pathology. While the basic configuration of vertebrae varies, every vertebra has a body that consists of a large anterior middle portion called the centrum and a posterior vertebral arch called the neural arch. The upper and lower surfaces of the vertebra body give attachment to intervertebral discs. The posterior part of a vertebra forms a vertebral arch that typically consists of two pedicles, two laminae, and seven processes. The laminae give attachment to the ligament flava, and the pedicles have a shape that forms vertebral notches to form the intervertebral foramina when the vertebrae articulate. The foramina are the entry and exit passageways for spinal nerves. The body of the vertebra and the vertical arch form the vertebral foramen, which is a large, central opening that accommodates the spinal canal that encloses and protects the spinal cord.

The body of each vertebra is composed of cancellous bone that is covered by a thin coating of cortical bone. The cancellous bone is a spongy type of osseous tissue, and the cortical bone is a hard and dense type of osseous tissue. The vertebral arch and processes have thicker coverings of cortical bone.

The upper and lower surfaces of the vertebra body are flattened and rough. These surfaces are the vertebral endplates that are in direct contact with the intervertebral discs. The endplates are formed from a thickened layer of cancellous bone, with the top layer being denser. The endplates contain adjacent discs and evenly spread applied loads. The endplates also provide anchorage for the collagen fibers of the disc.

FIG. 1 shows a portion of a patient's spinal column 2, including vertebrae 4 and intervertebral discs 6. As noted earlier, each disc 6 forms a fibrocartilaginous joint between adjacent vertebrae 4 so as to allow relative movement between adjacent vertebrae 4. Beyond enabling relative motion between adjacent vertebrae 4, each disc 6 acts as a shock absorber for the spinal column 2.

As noted earlier, each disc 6 comprises a fibrous exterior surrounding an inner gel-like center which cooperate to distribute pressure evenly across each disc 6, thereby preventing the development of stress concentrations that might otherwise damage and/or impair vertebrae 4 of spinal column 2. Discs 6 are, however, subject to various injuries and/or disorders which may interfere with a disc's ability to adequately distribute pressure and protect vertebrae 4. For example, disc herniation, degeneration, and infection of discs 6 may result in insufficient disc thickness and/or support to absorb and/or distribute forces imparted to spinal column 2. Disc degeneration, for example, may result when the inner gel-like center begins to dehydrate, which may result in a degenerated disc 8 having decreased thickness. This decreased thickness may limit the ability of degenerated disc 8 to absorb shock which, if left untreated, may result in pain and/or vertebral injury.

While pain medication, physical therapy, and other non-operative conditions may alleviate some symptoms, such interventions may not be sufficient for every patient. Accordingly, various procedures have been developed to surgically improve patient quality of life via abatement of pain and/or discomfort. Such procedures may include, discectomy and fusion procedures, such as, for example, anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF). During a discectomy, all or a portion of a damaged disc (for example, degenerated disc 8, shown in FIG. 1), is removed via an incision, typically under X-ray guidance.

Following the discectomy procedure, a medical professional may determine an appropriate size of an interbody device 10 (shown in FIG. 2) via one or more distractors and/or trials of various sizes. Each trial and/or distractor may be forcibly inserted between adjacent vertebrae 4. Upon determination of an appropriate size, one or more of an ACIF, ALIF, DLIF, PLIF, and/or TLIF may be performed by placing an appropriate interbody device 10 (such as, for example, a cage, a spacer, a block) between adjacent vertebrae 4 in the space formed by the removed degenerated disc 8. Placement of such interbody devices 10 within spinal column 2 may prevent spaces between adjacent vertebrae 4 from collapsing, thereby preventing adjacent vertebrae 4 from resting immediately on top of one another and inducing fracture of vertebra 4, impingement of the spinal cord, and/or pain. Additionally, such interbody devices 10 may facilitate fusion between adjacent vertebrae 4 by stabilizing adjacent vertebrae 4 relative to one another. Accordingly, as shown in FIG. 2, such interbody devices 10 often may include one or more bone screws 12 extending through interbody device 10 and into adjacent vertebrae 4.

Often, following the removal of the distractor and/or trial, a medical professional must prepare one or more bores or holes in a vertebra 4 intended to receive the bone screws 12. Such holes may be formed with the aid of a separate drill guide positioned proximate or abutting vertebra 4 and inserting a drill therethrough. Alternatively, such holes may be formed free hand, without the use of a drill guide. Further, since spinal column 2 is subject to dynamic forces, often changing with each slight movement of the patient, such screw(s) 12 have a tendency to back out (for example, unscrew) and/or dislodge from interbody device 10, thereby limiting interbody device's 10 ability to stabilize adjacent vertebrae 4, and consequently, promote fusion. Additionally, if screw(s) 12 back out and/or dislodge from the interbody device 10, they may inadvertently contact, damage, and/or irritate surrounding tissue. Further, interbody device 10 is commonly comprised of a radiopaque material so as to be visible in situ via x-ray and other similar imaging modalities. However, such materials may impede sagittal and/or coronal visibility, thereby preventing visual confirmation of placement and post-operative fusion.

Furthermore, while all metal titanium interbody devices 10 are good for bone ingrowth, they are radio-opaque and, thus, not good for monitoring bony fusion.

Thus, there remains a need for improved interbody devices, associated systems, and methodologies related thereto.

### SUMMARY OF THE DISCLOSURE

Accordingly, one aspect of the present disclosure provides a cage structure that can be made of different materials and textures. The cage structure may include various end surface textures with enhanced bone ingrowth while allowing for monitoring bony fusion.

According to an aspect of the present disclosure, an intervertebral cage structure is provided that comprises: a main body comprising a first surface and a second surface located opposite to the first surface; a plate disposed on the first surface of the main body; and an opening formed in the main body and extending from the first surface to the second surface located opposite the first surface, wherein the plate comprises a surface pattern having at least one of a symmetrical geometric pattern and an asymmetrical geometric pattern. The intervertebral cage structure may comprise a second plate disposed on the second surface of the main body. The main body may comprise Polyether Ether Ketone (PEEK). The plate may comprise titanium or a titanium alloy.

The main body may further comprise a plurality of lateral surfaces extending between the first and second surfaces; and one or more holes extending from one of the plurality of lateral surfaces towards the opening. The main body may further comprise an inner surface surrounding the opening. The inner surface may comprise a bulged portion surrounding a portion of the one or more holes.

The intervertebral cage structure may comprise a pin hole extending from the plate to the main body, and a pin that inserts into the pin hole.

The main body may further comprise one or more slots, and the plate may comprise one or more tabs that insert into the plurality of slots of the main body to secure the first plate to the main body. The plate may comprise a cutout that renders the plate compressible.

The intervertebral cage structure may comprise a shell main body, wherein the shell main body may be configured to receive and substantially encapsulate the main body. The shell main body may comprise a clam shape that includes said plate and the second plate, wherein said plate and the second plate are connected by a bridge portion. The main body may comprise at least one of a metal, PEEK, silicon and allograft.

According to another aspect of the disclosure, an intervertebral cage structure is provided that comprises: a shell main body having a clam shape and comprising a bridge portion and wing portions extending from the bridge portion; first and second surface layers disposed on the first and second wing portions; and an opening formed in the main body and extending from the first surface layer to the second surface layer. At least one of the first surface layer and the second surface layer may comprise at least one of a symmetrical geometric pattern and an asymmetrical geometric pattern. The shell main body may comprise PEEK and at least one of the first and second surface layers may comprise titanium or a titanium alloy.

The intervertebral cage structure may comprise an insertion. The insertion may be disposed between the first and second wing portions of the main body, wherein the opening may extend from the first surface layer to the second surface layer via the insertion. The insertion may comprise at least one of a metal, PEEK, silicon or allograft.

The intervertebral cage structure may comprise: a plurality of lateral surfaces extending between the first and second wing portions; and one or more holes extending from one of the plurality of lateral surfaces toward the opening.

The intervertebral cage structure may further comprise an inner surface surrounding the opening and having a bulged wall portion surrounding a portion of the one or more holes.

The intervertebral cage structure may include a slot and a guide that engages and guides the slot as the insertion is installed in the shell main body.

The intervertebral cage structure may further comprise: a plurality of lateral surfaces; and one or more screw holes extending from one of the plurality of lateral surfaces to the opening.

The intervertebral cage structure may further comprise first and second ears extending from the first and second wing portions, extending outwardly from each other, the first and second ears comprising one or more screw holes.

The surface pattern of the intervertebral cage structure may comprise first and second protrusions adjacent each other with a gap therebetween, wherein the first and second protrusions have an undercut at a lower portion thereof, wherein superior surfaces of the first and second protrusions may have different shapes, and wherein at least one of the first and second protrusions may have a pocket formed at the bottom surface thereof.

According to a further aspect of the disclosure, an intervertebral cage structure is provided that comprises a surface configured to contact a vertebra, the surface comprising first and second protrusions adjacent each other with a gap formed therebetween, the first and second protrusions having an undercut formed at a lower portion thereof. The superior surfaces of the first and second protrusions have different shapes. At least one of the first and second protrusions may have a pocket formed on the surface thereof.

Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 illustrates a portion of a patient's spinal column;

FIG. 2 illustrates an interbody device positioned within the patient's spinal column constructed according to the principles of the disclosure;

FIG. 3A illustrates a perspective view of an example of a cage structure that is constructed according to the principles of the disclosure;

FIG. 3B illustrates another view of the cage structure illustrated in FIG. 3A;

FIG. 4A illustrates an exploded view of the cage structure illustrated in FIGS. 3A and 3B;

FIG. 4B illustrates an example of an implant tool that may be used to install the cage structure;

FIG. 5A illustrates a perspective view of another example of a cage structure that is constructed according to the principles of the disclosure;

FIG. 5B illustrates another view of the cage structure illustrated in FIG. 5A;

FIG. 5C illustrates a superior (or inferior) view of the cage structure illustrated in FIGS. 5A and 5B;

FIG. 5D illustrates an anterior view of the cage structure illustrated in FIGS. 5A and 5B;

FIG. 5E illustrates a lateral view of the cage structure illustrated in FIGS. 5A and 5B;

FIG. 5F illustrates a posterior view of the cage structure illustrated in FIGS. 5A and 5B;

FIG. 5G illustrates a perspective anterior view of another example of a cage structure that is constructed according to the principles of the disclosure;

FIG. 6 illustrates an exploded view of the cage structure illustrated in FIGS. 5A and 5B;

FIG. 7A illustrates an enlarged cut view of an example of a surface pattern of the cage structure illustrated in FIG. 5A (or FIG. 3A, or FIG. 5G), constructed according to the principles of the disclosure;

FIG. 7B illustrates an enlarge cut view of another example of a surface pattern of the cage structure illustrated in FIG. 5A (or FIG. 3A, or FIG. 5G), constructed according to the principles of the disclosure;

FIG. 8A illustrates a perspective anterior view of an example of a shell, constructed according to the principles of the disclosure;

FIG. 8B illustrates a lateral view of the shell illustrated in FIG. 8A;

FIG. 8C illustrates a perspective anterior view of another example of a shell, constructed according to the principles of the disclosure;

FIG. 8D illustrates a lateral view of a further example of a shell, constructed according to the principles of the disclosure;

FIGS. 9A and 9B illustrate anterior and lateral views of an example of a shell of the cage structure illustrated in FIG. 5A;

FIG. 10A illustrates an exploded view of another example of a cage structure that is constructed according to the principles of the disclosure;

FIG. 10B illustrates another view of the cage structure illustrated in FIG. 10A;

FIG. 10C illustrates an exploded view of a further example of a cage structure that is constructed according to the principles of the disclosure;

FIG. 10D illustrates another example of an insertion, constructed according to the principles of the disclosure;

FIGS. 10E and 10F illustrate perspective anterior and lateral views, respectively, of another example of a cage structure constructed according to the principles of the disclosure;

FIG. 11A illustrates an example of another cage structure, constructed according to the principles of the disclosure; and

FIG. 11B illustrates the cage structure shown in FIG. 11A, which is inserted between two adjoining vertebrae.

### DETAILED DESCRIPTION OF THE DISCLOSURE

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

The terms “including,” “comprising” and variations thereof, as used in this disclosure, mean “including, but not limited to,” unless expressly specified otherwise.

The terms “a,” “an,” and “the,” as used in this disclosure, mean “one or more,” unless expressly specified otherwise.

Devices that are in communication with each other need not be in continuous communication with each other, unless expressly specified otherwise. In addition, devices that are in direct contact with each other may contact each other directly or indirectly through one or more intermediary articles or devices. The device(s) disclosed herein may be made of a material such as, for example, a polymer, a metal, an alloy, or the like. For instance, the device(s) may be made of Polyether Ether Ketone (PEEK), titanium, a titanium alloy, or the like, or a combination of the foregoing. The material may be formed by a process such as, for example, an active reductive process of a metal (e.g., titanium or titanium alloy) to increase the amount of nanoscaled texture to device surface(s), so as to increase promotion of bone growth and fusion.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device or article may be alternatively embodied by one or more other devices or articles which are not explicitly described as having such functionality or features.

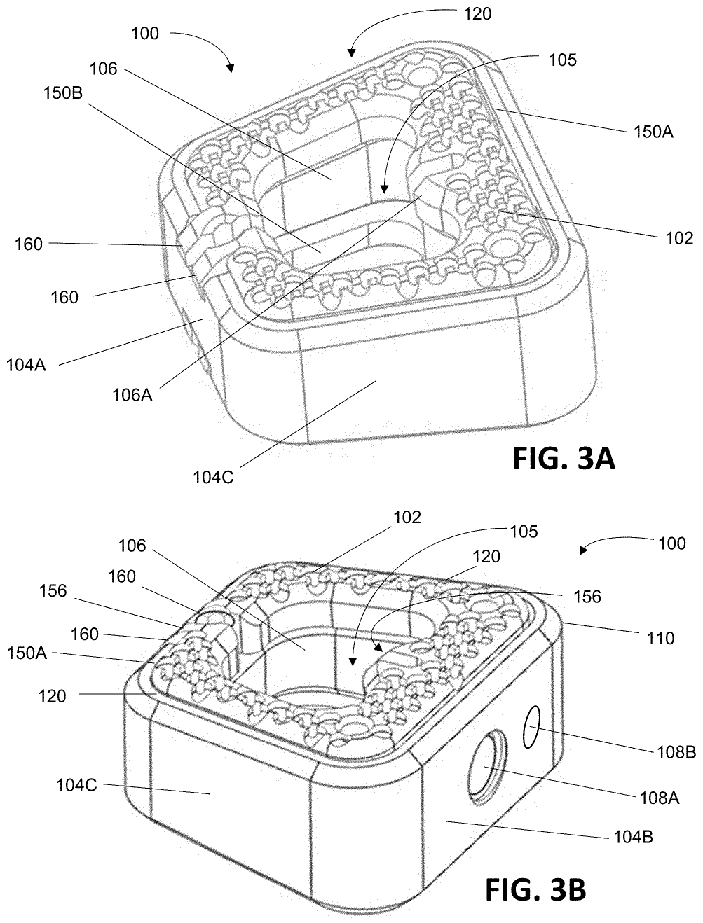

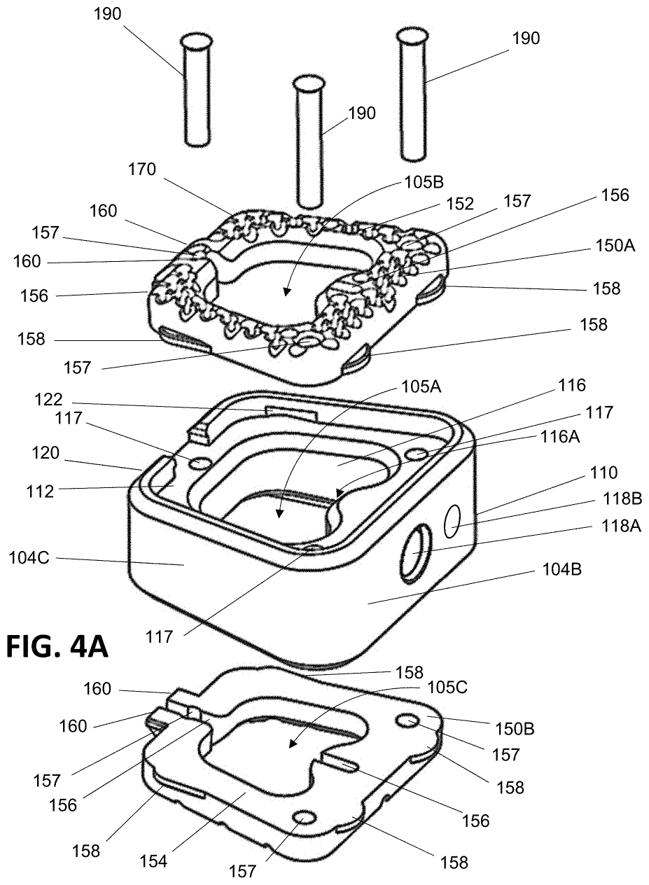

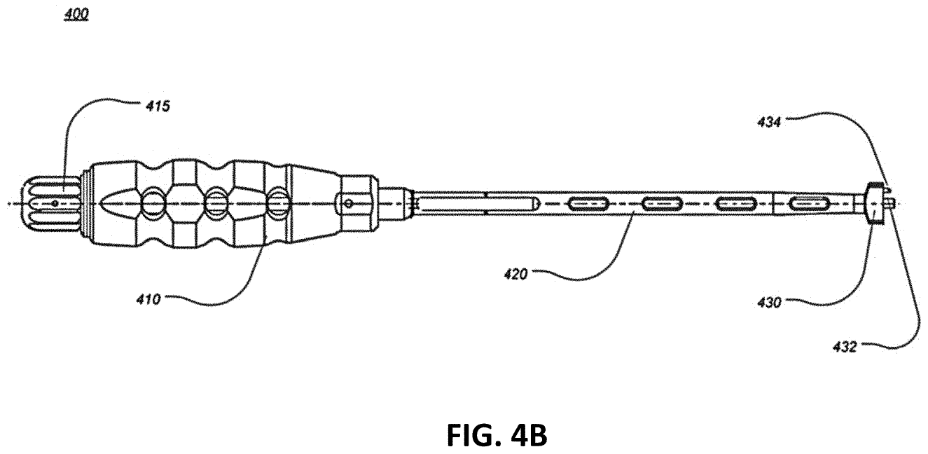

FIGS. 3A through 4A illustrate various views of a cage structure 100 that is constructed according to the principles of the disclosure, with FIG. 3A illustrating a perspective view of a cage structure 100; FIG. 3B illustrating another view of the cage structure 100; and FIG. 4A illustrating an exploded view of the cage structure 100. The cage structure 100 may be constructed as one, two, three, or more parts. The cage structure 100 may be made of a material such as, for example, a polymer, a metal, an alloy, or the like. For instance, the cage structure 100 may be made of PEEK, titanium, a titanium alloy, or the like. The surfaces of the cage structure 100 may be formed to increase the amount of nanoscaled texture to increase promotion of bone growth and fusion in the implant area, wherein the formation may include forming a surface by, for example, an active reductive process of, e.g., titanium or titanium alloy.

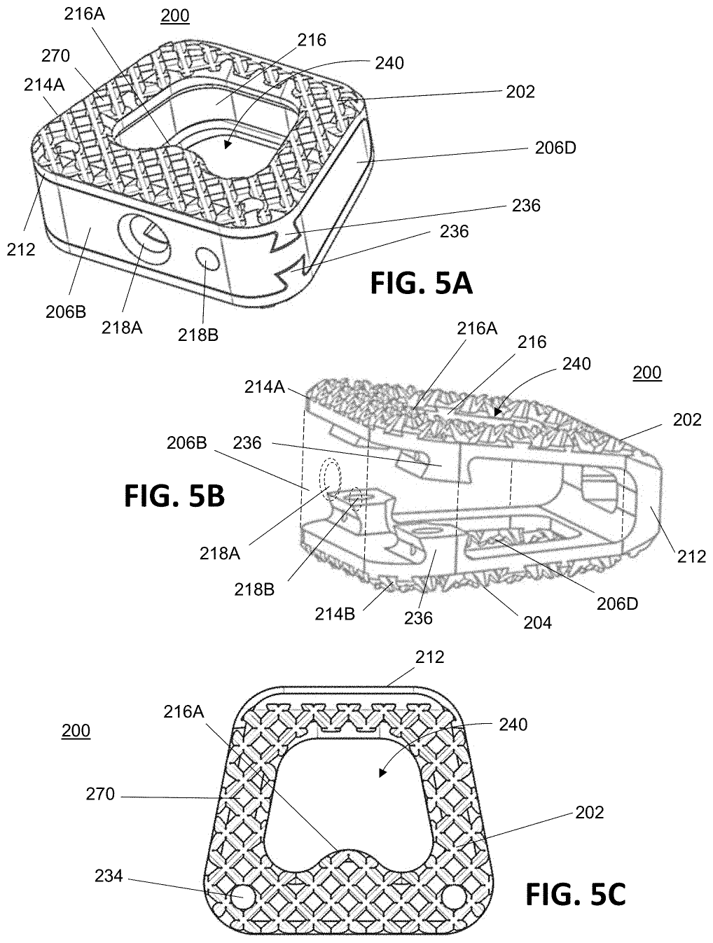

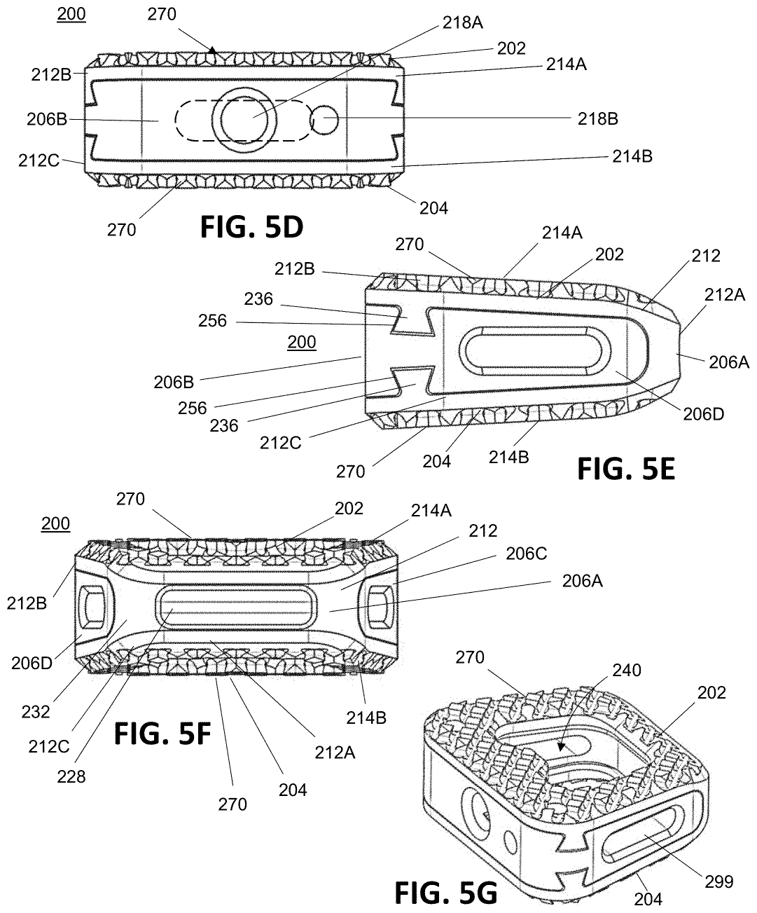

Referring to FIGS. 3A and 3B, in an embodiment of the cage structure 100 that has only one part (which could include an embodiment similar to that shown in FIG. 5G), the cage structure 100 may comprise only the main body 110. In this embodiment, the main body 110 may be formed as a single piece with a first main surface 102 on one side of the main body 110 (as seen in FIGS. 3A and 3B) and a second (opposite) main surface (not shown) on the other side of the main body 110. The cage structure 100 may be implanted standalone or with a supplementary fixation device such as, for example, a plate (e.g., anterior cervical plate), a bone fastener(s), and/or the like.

Referring to FIGS. 3A through 4A concurrently, in an embodiment of the cage structure 100 that has two or more parts, the cage structure 100 may include the main body 110 and one or more plates 150A (and/or 150B). The cage structure 100, which may have the first main surface 102 and the second main surface (not shown) located opposite to the first main surface 102, may directly contact two adjacent vertebrae, respectively, when the cage structure 100 is inserted therebetween. The first main surface 102 may be provided on the plate 150A (or 150B). The second main surface (not shown) may be provided on the plate 150B (or 150A).

In the cage structure 100, the first main surface 102 may include a surface pattern such as, for example, the surface pattern shown in FIG. 7A or 7B and described in detail below, or any other pattern that may assist in capturing and retaining blood, tissue, bone graft, or the like, to promote bone growth or fusion. The second main surface (not shown) may have the same or a different surface pattern as the first main surface 102. The surface pattern may include, for example, sharp teeth on the surface to ensure primary stability and prevent migration of the cage structure 100. The surface pattern may be configured (e.g., as shown in FIG. 7A or 7B) to promote integration and bone ongrowth and ingrowth within the roughened surface for good stability.

The surface pattern may be provided on any surface area, including that of a cage structure (e.g., cage structure 100), where bone cells can attach and grow, including, for example, external sagittal walls, external coronal walls (front and/or back), and the like. The surface pattern may be provided to any cage shape or form with, or without supplementary fixation features, including, for example, cages shapes/forms configured for ACIF, PLIF, TLIF, DLIF, OLIF, VBR, and the like.

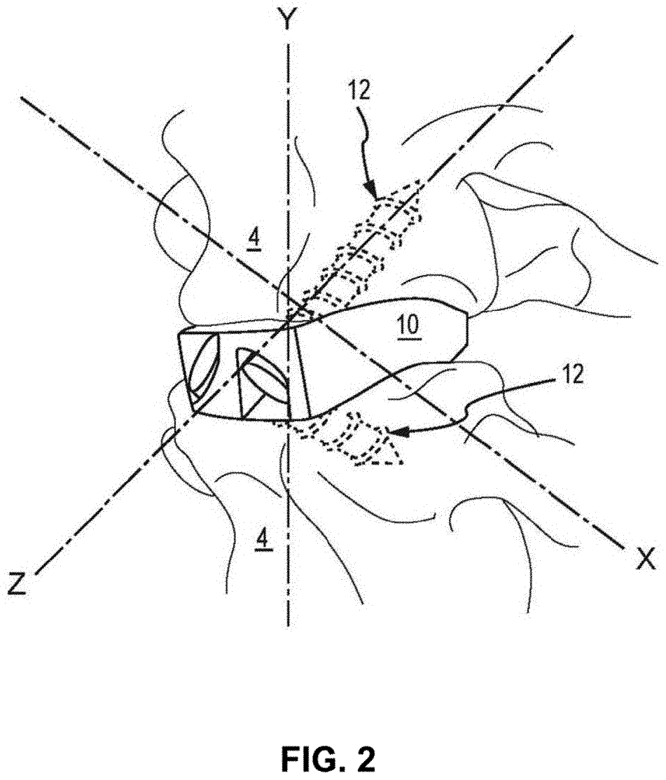

The cage structure 100 may be configured to have a shape in a horizontal plane in the form of, for example, a rectangle, a trapezoid, a square, a pentagon, a circle, an oval, a hexagon, or any other shape that may be appropriate for a particular application, as understood by those skilled in the art. The cage structure 100 may be formed to substantially match the shape and/or size of the space between the adjacent vertebrae, as well as the shape and size of the vertebrae surfaces (e.g., vertebra 4 shown in FIG. 2) that contact the first main surface 102 and opposing second main surface (not shown) of the cage structure 100, when the cage structure 100 is implanted. The cage structure 100 may have a substantially wedge-shaped design to accommodate endplate shape variances. In the vertical plane (i.e., the plane perpendicular to the horizontal plane), the cage structure 100 may have different heights for the anterior and posterior portions of the cage structure 100, so as to properly fill the space between the adjacent vertebrae.

The cage structure 100 may include a plurality of side wall surfaces 104 that may extend between the first main surface 102 and the second main surface (not shown). The side wall surfaces 104 and the first and second main surfaces may form the outer shape of the cage structure 100. The plurality of side wall surfaces 104 may include, for example, a posterior wall surface 104A, an anterior wall surface 104B, and a pair of lateral (or side) wall surfaces 104C located opposite each other.

The cage structure 100 may include an opening 105. The opening 105 may be formed in or near the center portion of the cage structure 105. The opening 105 may extend between the superior and inferior directions of the cage structure 100, extending from the first main surface 102 to the second main surface (not shown). The opening 105 may be defined and laterally surrounded by inner wall surface(s) 106 of the cage structure 100. The opening 105 may form a chamber, such as, for example, a graft chamber that is configured to receive, for example, blood, tissue, bone, bone graft and the like, to promote bone growth or fusion. The inner wall surfaces 106 may have a surface pattern (not shown) that may help in retaining blood, tissue, bone graft, etc., in the graft chamber.

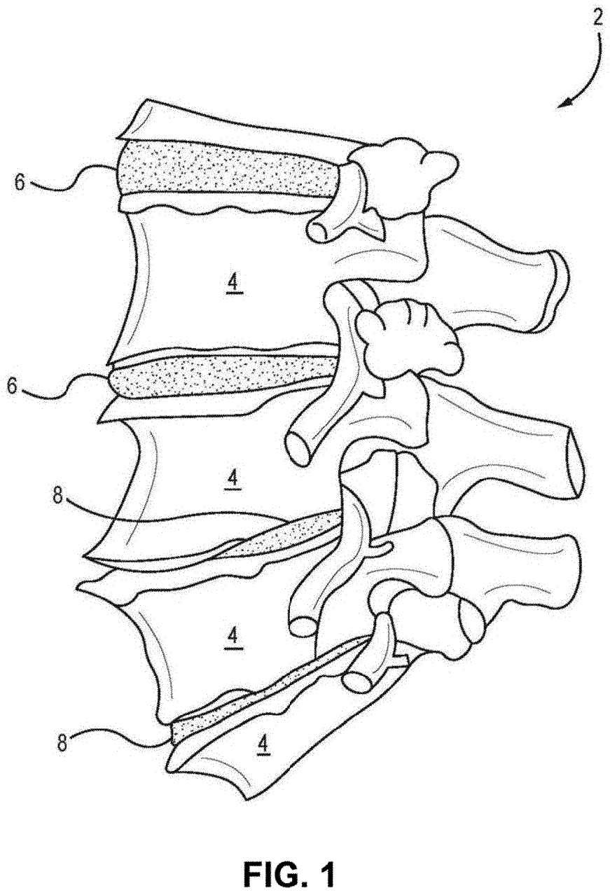

The cage structure 100 may include one or more openings or windows (not shown), such as, for example, window(s) 299 shown in FIG. 50. The window(s) may be formed in the lateral, posterior and/or anterior walls. Such windows may remain empty and/or may be filled with radiolucent material such as tissue grafts as will be described in further detail below. The windows may enable a medical professional to view and/or determine the level of post-operative fusion between cage structure 100 and patient bone and/or tissue. The cage structure body may define any appropriate arrangement, number, and configuration of windows. As seen in the example in FIG. 5G, for example, the cage structure 100 may include a pair of windows 299 on each lateral side. Each window may be generally quadrilateral (e.g., square, rectangular, or trapezoidal). In some arrangements, a radiolucent structure, such as a graft containment sheath, may be disposed along one or more portions of cage structure 100. Indeed, such graft containment sheaths may substantially fill or encompass window. Accordingly, when the cage structure 100 is placed between two adjacent vertebrae 4 (shown in FIG. 1) under X-ray vision, the window remains radiolucent such that fusion within and/or through window may be observed.

As seen in FIGS. 3B and 4A, the cage structure 100 may include one or more holes (or openings), such as, for example, a hole 108A and a hole or recessed portion 108B. Alternatively (or additionally), the cage structure 100 may include fastening holes (not shown) that may be configured to receive one or more bone fasteners (e.g., bone screws 12 shown in FIG. 2) to secure the cage structure 100 to adjacent vertebra. In this regard, the fastening holes (not shown) may be angled so as to guide the bone fasteners toward and into the adjacent vertebrae. FIG. 2 shows an example of fastening holes formed in an implantable device and angled so as to guide the bone screws 12 toward and into adjacent vertebrae 4.

FIG. 4B shows an example of an implant tool 400 that may be used to install the cage structure 100 in a spinal column of a patient. The implant tool 400 includes a handle 410, a shaft 420, and a contact head 430. The handle 410 includes an engaging member 415 that is connected to or integrally formed with an internal shaft (not shown) that has a threaded end 432. The internal shaft (not shown) may be housed in the shaft 420. The threaded end 432 of the internal shaft may protrude from the contact head 430, as seen in FIG. 4B. The contact head 430 may include an orientation guide 434 (such as, for example, an orientation peg). The orientation guide 434 may be integrally formed with the contact head 430.

Referring to FIGS. 3A-4A concurrently, the cage structure 100 (with or without a plating device (not shown)) may be configured for use in, for example, anterior approach and discectomy applications. For instance, after a surgical area is cleaned on a patient, an incision made, muscle tissue and/or organs moved to the side(s), and other common surgical procedures carried out, a disc may be incised, removed, and the space prepared for implanting of the cage structure 100. The bone surfaces and edges on the adjacent vertebrae may be carefully contoured, as appropriate.

Following a discectomy procedure, a medical professional may determine an appropriate size of the cage structure 100 by selecting an appropriately dimensioned cage structure 100 and an appropriately dimensioned plating device (not shown), if applicable, which may be selectable based on, for example, height, width, depth, and the like. Upon selecting the appropriate cage structure 100 (and plating device, if applicable), one or more of an ACIF, ALIF, PLIF, TLIF, DLIF, OLIF, VBR, or the like may be performed by placing the cage structure 100 between adjacent vertebrae 4 in the space formed by the removed degenerated disc. Placement of the cage structure 100 within the spinal column may prevent spaces between adjacent vertebrae 4 from collapsing, thereby preventing adjacent vertebrae from resting immediately on top of one another and inducing fracture of vertebra 4, impingement of the spinal cord, and/or pain. Additionally, such cage structures 100 may facilitate fusion (e.g., bone to grow together) between adjacent vertebrae 4 by stabilizing adjacent vertebrae 4 relative to one another and promoting bone ingrowth.

Referring to FIGS. 3A-4B, the implant tool 400 may be securely connected to the cage structure 100 by aligning the threaded end 432 and the orientation guide 434 with the holes 108A and 108B, respectively. The threaded end 432 may be inserted in and turned by manipulating the engagement member 415 to engage a corresponding threading in the hole 108A, thereby securing the cage structure 100 to the contact head 430. The orientation guide 434 may be inserted in the hole 108B, so as to properly align the implant tool 400 with respect to the cage structure 100, while preventing the cage structure 100 from rotating with respect to the contact head 430.

The hole 108A may be located, for example, at the center of the wall surface 104B. The hole 108A may have a larger diameter than the hole 108B. The hole 108A may be threaded to engage the threaded end 432 of the implant tool 400. The hole 108B may be constructed to engage the orientation guide 434 of the implant tool 400. The hole 108A may be deeper than the hole 108B.

Once the implant tool 400 is securely and fixedly attached to the cage structure 100, the surgeon may align and implant the cage structure 100 in the space prepared for implanting of the cage structure 100. If applicable, the surgeon may implant a plating device (not shown), which may be secured to the adjacent vertebrae 4, as is known by those skilled in the art. After the cage structure 100 is properly positioned in the space between the vertebrae 4, the surgeon may release the cage structure 100 by turning the engaging member 415 in the opposite direction to unthread the threaded end 432.

The cage structure 100 may include a wall portion 106A that may be bulged inwardly to provide added strength for the area surrounding the hole 108A, so as to be able receive and withstand substantial force that may be applied to the cage structure 100 through the implant tool 400.

Referring to FIG. 4A, the cage structure 100 may be constructed with two or more parts, including the main body 110 and one or more plates 150A, 150B. The cage structure 100 may further include one or more fasteners (e.g., pins 190A, 190B, 190C) to secure the one or more plates 150A, 150B to the main body 110.

The main body 110 and the first and second plates 150A, 150B may be formed of one or more robust, strong and ductile materials, such as, for example, a polymer, a metal, an alloy, or the like. For example, the main body 110 may be formed of PEEK, and the first and second plates 150A, 150B may be formed of titanium or a titanium alloy. The main body 110 and the first and second plates 150A, 150B may be a single unitary piece or an assembly of two or more parts that are independently produced.

As seen in FIG. 4A, the main body 110 may have a first surface 112 (shown facing upwardly) and a second surface (not shown) located opposite to the first surface 112 and facing in the opposite direction. Side surfaces of the main body 110 may be exposed, and the wall surfaces 104A, 104B, 104C of the cage structure 100 may be the side wall surfaces of the main body 110. The anterior wall surface 104B may be wider than the posterior wall surface 104A, and the first main surface 102 and the second main surface (not shown) may have a generally trapezoidal shape with rounded corners. The anterior wall surface 104B may be thicker (or wider) than the posterior wall surface 104A, and the side or lateral wall surfaces 104C may have a generally trapezoidal shape.

The first and second plates 150A, 150B may be attached to the first surface 112 and the second surface (not shown) of the main body 110, respectively. The main body 110 may be vertically and/or horizontally symmetric, in which case the first surface 112 may be configured to contact either or both of the surfaces of the first and second plates 150A, 150B. The first and second plates 150A, 150B may have substantially the same shape and construction, and hence may be interchangeably used. Alternatively, the first surface 112 and the second surface (not shown) of the main body 110 may have different shapes and constructions; and, the first and second plates 150A, 150B may be shaped and constructed differently to fit to the first surface 112 and the second surface, respectively.

The main body 110 may have an opening 105A (shown in FIG. 4A) extending from the first surface 112 to the second surface (not shown) of the main body 110. The opening 105A may be located, for example, at or near the center of the main body 110. The opening 105A may be defined by an inner wall surface 116 of the main body 110. The holes 108A, 108B may be formed in the main body 110, and the inner surface 116 may have a bulged portion 116A to provide added strength and stability around the hole 108A. The first and second plates 150A, 150B may have openings 105B, 105C, respectively, which may be formed corresponding to the opening 105A. A retention member (not shown), such as, for example, a mesh, a grid, or the like, may be formed in the openings 105B and/or 105C, so as to retain a bone graft material in the opening 105A. The retention member should have a structure, so as to promote fusion and bone growth between the bone graft material and the adjacent vertebra. The openings 105A. 105B, 105C may collectively form the opening 105 (shown in FIGS. 3A and 3B).

As seen in FIG. 4A, the first and second plates 150A and 150B may have an outer surface 152 (shown with the first plate 150A) and an inner surface 154 (shown with the second plate 150B). The inner surface 154 may be substantially flat and smooth. The first surface 112 and the second surface (not shown) of the main body 110 may be substantially flat and smooth. The inner surfaces 154 may be in direct contact with the first surface 112 and the second surface (not shown) of the man body 110.

The first and second plates 150A, 150B may be attached to the main body 110 by an adhesive, a fastener, or the like. For example, the first plate 150A may be adhered to or snapped in the main body 110. Alternatively or additionally, the first and second plates 150A, 150B may be attached to the main body 110 by one or more fasteners, such as, for example, a pin, a screw, a rivet, a bolt, a nut, or the like. For example, the main body 110 may include one or more pin holes 117 (three shown in FIG. 4A). The first plate 150A may have one or more pin holes 157 (three shown in FIG. 4A), which may be aligned with the pin holes 117 of the main body 110. One or more pins 190 (three shown in FIG. 4A) may be driven into the pin holes 157 and the pin holes 117 to attach the first plate 150A on the first surface 112 and/or the second plate 150B of the main body 110. The pins 190 may be radiopaque or radiolucent.

Alternative or additionally, the main body 110 and the first and second plates 150A, 150B may be constructed to structurally engage each other. For example, the first surface 112 of the main body 110 may have a wall 120 protruding upwardly and extending along a periphery of the first surface 112. As seen in FIGS. 3A and 3B, the wall 120 may surround the first plate 150A such that the first plate 150A may not move around laterally.

Additionally, the main body 110 may have one or more recesses 122, and the first and second plates 150A, 50B may have one or more tabs 158, which may be located and shaped to fit into the recesses 122 of the main body 110. For example, as seen in FIG. 4A, a pair of tabs 158 may be formed at a posterior edge of the first plate 150A, and another pair of tabs 158 may be formed at right and left sides of the first plate 150A, respectively. The main body 110 may have four recesses 122 (only one shown in FIG. 4A). A pair of recesses 122 may be formed at the wall 120 on a posterior portion of the main body 110. Another pair of recesses 122 may be formed at the wall 120 on right and left portions of the main body 110, respectively. Thus, the first and/or second plates 150A, 150B may be snapped into and held securely in position with respect to the main body 110.

The first plate 150A may have one or more cutouts 156 (two shown) and one or more push tabs 160 (more clearly shown with the second plate 150B in FIG. 4A). The cutouts 156 may be positioned to render the first plate 150A compressible. The push tabs 160 may be formed at a posterior portion of the first plate 150A. The push tabs 160 may be pushed (or squeezed) toward each other to compress the first plate 150A, which may result in inwardly retracting the tabs 158 on the right and left sides of the first plate 150. Once the compressed first plate 150A is placed on the first surface 112, the push tabs 160 may be let go to decompress the first plate 150A, and the tabs 158 may be inserted and fit into the corresponding recesses 122, respectively. Once the tabs 158 are inserted into the recesses 122, the first plate 150A may not move vertically or horizontally. As seen in FIG. 4A, the wall 120 may be discontinued at a posterior portion of the main body 110 where the push tabs 158 are placed. The second plate 150B may be constructed in a similar manner and attached to the main body 110 in a similar manner.

The outer surface 152 of the first and second plates 150A, 150B may have a surface pattern 170 that may form the first main surface 102 and/or the second main surface (not shown). The surface pattern 170 may establish and promote bone growth and resist movement (e.g., departure, slippage, etc.) installed with respect to a vertebra. The surface pattern 170 may include a symmetrical geometric pattern (e.g., circle, sphere, semi-sphere, equilateral triangle, pyramid, isosceles triangle, square, rectangle, kite, rhombus, pentagon, hexagon, heptagon, octagon, or the like), an asymmetrical geometric pattern (e.g., irregular sphere or semi-sphere, scalene triangle, irregular pyramid, irregular quadrilateral, irregular pentagon, irregular hexagon, irregular heptagon, irregular octagon, or the like), a combination of one or more symmetrical geometric patterns and/or one or more asymmetrical geometric patterns, and/or the like. The surface pattern 170 may be formed by, for example, machining, chemically machining, and/or stamping the outer surface 152. Alternatively or additionally, the outer surface 152 may be chemically processed by performing micro-surface treatments, such as, for example, chemical etching, hydroxyapatite coating, and/or the like. The surface pattern 170 may have a structure shown in FIG. 7A or 7B and described below.

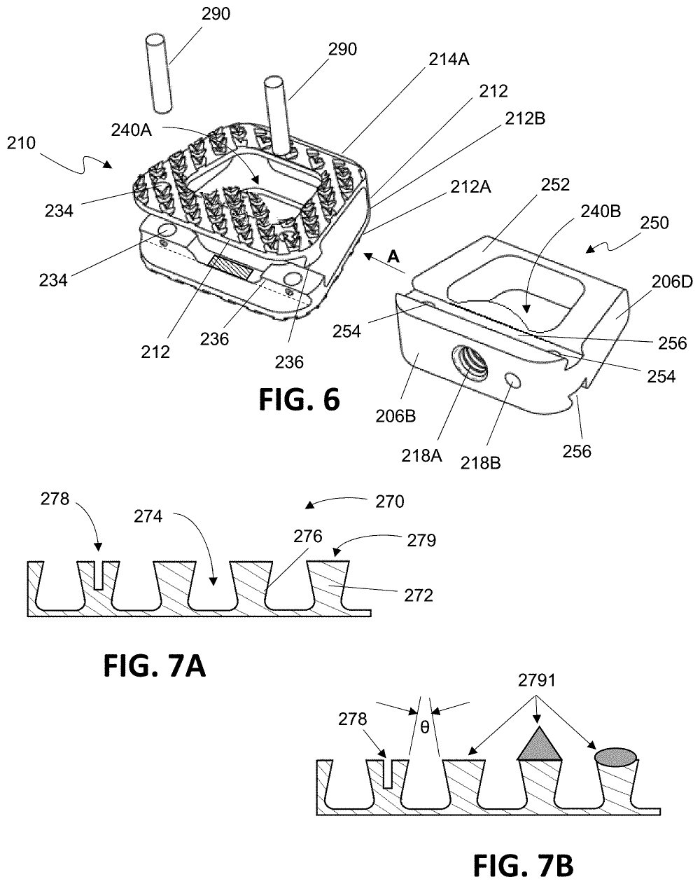

FIGS. 5A-5F and 6 illustrate various views of another cage structure 200 that is constructed according to the principles of the disclosure. FIG. 5A illustrates a perspective view of the cage structure 200; FIG. 5B illustrates another perspective view of the cage structure 200; FIGS. 5C, 5D, 5E, 5F illustrate superior (or inferior), anterior, lateral and posterior views of the cage structure 200, respectively; and FIG. 6 illustrates an exploded perspective view of the cage structure 200.

FIG. 5G illustrates yet another example of a cage structure 200′ that is constructed according to the principles of the disclosure.

FIG. 7A illustrates a side cut view of a surface pattern of the cage structure 200 (or the cage structure 100 shown in FIGS. 3A-4A, or the cage structure 200′ shown in FIG. 5G). FIG. 7B illustrates a side cut view of another example of a surface pattern of the cage structure 200 (or the cage structure 100 shown in FIGS. 3A-4A, or the cage structure 200′ shown in FIG. 5G).

Referring FIGS. 5A-5F, and 6-7A concurrently, the cage structure 200 may have a first surface 202 (shown facing upwardly) and a second surface 204 (shown facing downwardly) located opposite to the first surface 202, and a plurality of side surfaces (e.g., a posterior surface 206A, an anterior surface 206B, and lateral surfaces 206C and 206D). The anterior surface 206B may be wider and thicker than the posterior surface 206A. Hence, as seen in FIG. 5C, the first surface 202 (and the second surface 204) may have a generally trapezoidal shape with rounded corners in the lateral (or horizontal) plane. Also, as seen in FIG. 5E, the lateral surfaces 206C and 206D may be tapered from the anterior surface 206B to the posterior surface 206A. The cage structure 200 may be vertically symmetric, and may be turned over vertically when inserted into a body of a patient. The cage structure 200 may be horizontally symmetric.

As seen in FIGS. 5A, 5B, 5D and 6, the cage structure 200 may include one or more holes (or openings), such as, for example, a hole 218A and a hole 218B. Alternatively (or additionally), the cage structure 100 may include fastening holes (not shown) that may be configured to receive one or more bone fasteners (e.g., bone screws 12 shown in FIG. 2) to secure the cage structure 200 to adjacent vertebra. In this regard, the fastening holes (not shown) may be angled so as to guide the bone fasteners toward and into the adjacent vertebrae. FIG. 2 shows an example of fastening holes formed in an implantable device and angled so as to guide the bone screws 12 toward and into adjacent vertebrae 4.

Referring to FIGS. 5A, 5B, 5D, and 6, the holes 218A, 218B may extend inwardly from the anterior surface 206B to engage, for example, the implant tool 400 (shown in FIG. 4B) or the like. For example, similar to the holes 108A, 108B of the cage structure 100, the holes 218A, 218B may be constructed to engage the threaded end 432 of the inner shaft and an orientation guide, respectively, of the implant tool 400, shown in FIG. 4B. The cage structure 200 may be implanted in a patient in substantially the same manner as the cage structure 100, described above.

The cage structure 200 may include an opening 240, which may extend from the first surface 202 to the second surface 204. The opening 240 may be a graft chamber, or the like, similar to the opening 105 (shown in FIGS. 3A and 3B) discussed above. As seen in FIG. 5C, the opening 240 may be formed at, for example, a center portion of the cage structure 200. The opening 240 may be laterally surrounded and defined by an inner wall surface 216. The inner wall surfaces 216 may have a wall portion 216A that may bulge inwardly to provide added strength for the area surrounding the hole 218A, so as to be able receive and withstand substantial force that may be applied to the cage structure 200 through the implant tool 400.

The first and second surfaces 202, 204 may have a surface pattern 270, which may be configured to directly contact a surface of the adjacent vertebra during implantation. The surface pattern 270 may establish and promote bone growth and resist movement (e.g., departure, slippage, or the like).

As seen in FIGS. 7A and 7B, the surface pattern 270 may include a plurality of protrusions 272 with a plurality of gaps 274 therebetween. A bottom portion of the protrusions 272 may be caved in with each lateral inner wall of adjacent protrusions 272 formed at an angle .theta. (shown in FIG. 7B) with respect to the normal axis of the surface pattern 270, thereby forming an undercut 276 that enlarges a bottom portion of the gaps 274. The angle .theta. may range anywhere from 0.degree. and 45.degree. However, the angle .theta. may be less than 0.degree. or greater than 45.degree. with respect to the normal axis. The gap 274 enlarged by the undercut 276 may function as a bone lock post, which may promote bone fusion and growth.

The protrusions 272 may include a pocket 278, which may be a hole or a slot formed at a superior (or inferior) surface 279 thereof, to increase a bone growth area. The superior surfaces 279 may have one or more symmetric geometry shapes, one or more asymmetric geometry shapes, a combination of a symmetric geometry shape and an asymmetric geometry shape, or the like. Two neighboring protrusions 272 may have different superior surface shapes. FIG. 7B shows an example wherein one of the two neighboring protrusions 272 may have a triangular or pyramid-shaped superior surface 2791 and the other may have a circular or semi-spherical-shaped superior surface 2791. The protrusions 272 with different surface shapes may be arranged alternatingly.

FIG. 5G shows another example of a cage structure 200′ that is constructed according to the principles of the disclosure. The cage structure 200′ may be made entirely of a metal (e.g., titanium) or metal alloy (e.g., titanium alloy). The cage structure 200′ may be formed as a single piece or may be of modular and/or multi-piece construction, having first and second surfaces 202, 204, with either or both surfaces having the surface pattern 270. As seen, the cage structure 200′ may include one or more openings or windows 299. Such windows 299 may remain empty and/or may be filled with radiolucent material such as tissue grafts as will be described in further detail below. Window(s) 299 may enable a medical professional to view and/or determine the level of post-operative fusion between cage structure 200′ (or 200) and patient bone and/or tissue. The cage structure 200′ body may define any appropriate arrangement, number, and configuration of windows 299. That is, as shown in FIG. 5G, for example, the cage structure 200′ may include a pair of windows 299 on each lateral side. Each window 299 may be generally quadrilateral (e.g., square, rectangular, or trapezoidal). In some arrangements, a radiolucent structure, such as a graft containment sheath, may be disposed along one or more portions of cage structure 200′. Indeed, such graft containment sheaths may substantially fill or encompass window 299. Accordingly, when the cage structure 200′ is placed between two adjacent vertebrae 4 (shown in FIG. 1) under X-ray vision, window 299 remains radiolucent such that fusion within and/or through window 299 may be observed.

As seen in FIG. 6, the cage structure 200 may be constructed as one, two, or more parts. The cage structure 200 may be constructed as a shell 210 and/or an insertion (or main body) 250. The cage structure 200 may further include one or more fasteners (e.g., pins 290). The shell 210 may have an opening 240A formed at a center portion. The shell 210 may be constructed as a single piece that includes only the shell 210 or insertion 250, or with two or more pieces that are assembled together, including the shell 210 and insertion 250. The insertion 250 may include one or more windows, such as, for example, window 299 shown in FIG. 5G and described above.

For example, as seen in FIG. 5E, the shell 210 may be constructed with a shell main body 212 and one or more surface layers 214A, 214B. The shell main body 212 may have a generally clam shape (or U-shape). The shell main body 212 may include a bridge portion 212A and a pair of wing portions 212B, 212C extending from two opposite sides of the bridge portion 212A. As seen in FIG. 5F, the bridge portion 212A may form the anterior surface 206A. The bridge portion 212A may include an opening 228. The opening 228 may function to allow blood, tissue, bone graft, etc., to flow into (or out from) the shell 210.

The surface layers 214A, 214B may be attached to outer surfaces of the wing portions 212B, 212C, respectively, or the surface layers 214A, 214B may be integrally formed with the wing portions 212B, 212C. The surface layers 214A, 214B may include the first and second surfaces 202, 204, respectively. Inner surfaces of the bridge portion 212A and the wing portions 212B, 212C may be smooth and clean to reduce friction when the insertion 250 is inserted to a space surrounded by the shell 210.

The shell main body 212 may be formed of one or more materials that may provide a visible fusion window. For example, the shell main body 212 may be formed of PEEK or the like. The surface layers 214A, 214B may be formed of one or more materials that can be processed to form the surface pattern 270 having, for example, undercut 276, pocket 278, and/or the like. For example, the surface layers 214A, 214B may be formed of titanium, a titanium alloy, or the like.

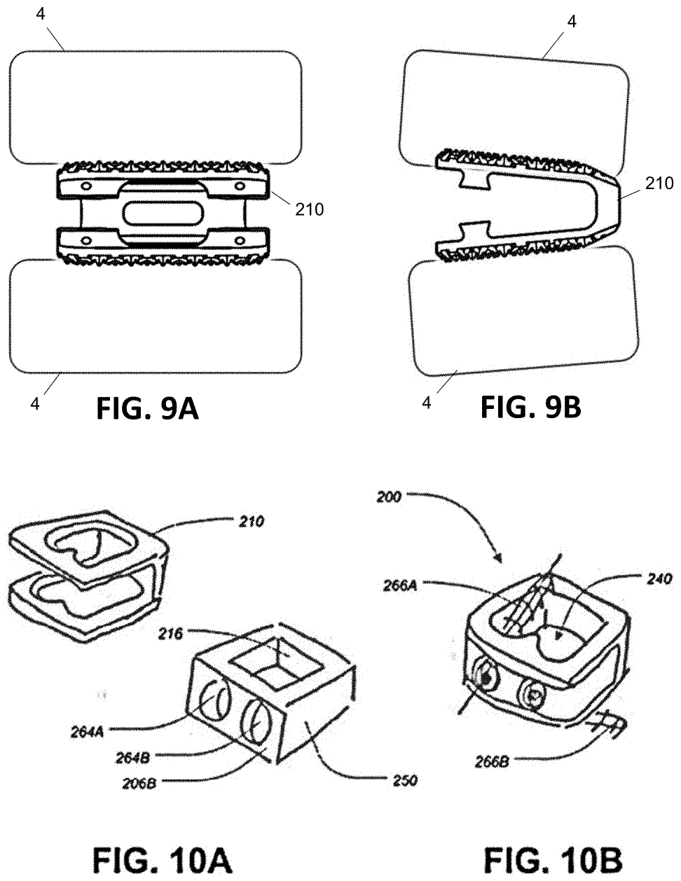

The shell 210 of the cage structure 200 may be used alone as a cage, without any other parts. For example, as seen in FIGS. 9A and 9B, the shell 210 may be inserted between adjacent vertebrae 4 without the insertion 250. Similarly, the insertion 250 may be used alone as a cage, without any other parts (not shown).

The insertion 250 may be constructed to fit into a space surrounded by the shell 210. As seen in FIG. 6, the insertion 250 may have a plurality of surfaces, and some of the surfaces may form the posterior surface 206B, and the lateral surfaces 206C and 206D of the cage structure 200. Other surfaces, such as, for example, first insertion surface 252, second insertion surface (not shown) located opposite to the first insertion surface 252, anterior insertion surface (not shown) opposite to the posterior surface 206B, and the like, may be covered and/or encapsulated by the shell 210 and may not be visible. The anterior insertion surface (not shown) may be partially exposed by the opening 228 located at the anterior surface 206A of the cage structure 200. An opening 240B may be formed at a center portion of the insertion 250. The openings 240A and 240B may collectively form the opening 240 of the cage structure 200.

The insertion 250 may be formed of metal (e.g., titanium, a titanium alloy, or the like), a radiopaque or radiolucent material (e.g., PEEK), an elastic and/or shock-absorbing material (e.g., silicon), an allograft bone, or the like. The insertion 250 may be a single unitary piece or a combination of multiple pieces that are manufactured separately. As noted earlier, the insertion 250 may include one or more windows, such as, for example, window 299 shown in FIG. 5G and described above.

The shell 210 and the insertion 250 may be assembled together by an adhesive, a fastener, or the like. For example, the shell 210 and the insertion 250 may be glued together. Alternatively or additionally, the shell 210 may be attached to the insertion 250 by one or more fasteners, such as, for example, a pin, a screw, a rivet, a bolt, a nut, or the like.

For example, as seen in FIGS. 5C and 6, the shell 210 may have one or more pin holes 234 (e.g., two) formed at an anterior (or posterior) portion of the first surface 202. The insertion 250 may also one or more pin holes 254 formed at an anterior (or posterior) portion of the first insertion surface 252. The pin holes 234 and 254 may be aligned when the shell 210 and the insertion 250 are put together. One or more corresponding pins 290 may be inserted into the pin holes 234 and 254 to affix the shell 210 to the insertion 250. The pins 290 maybe radiopaque or radiolucent.

The shell 210 and the insertion 250 may be constructed to mate to each other and form a unitary structure. For example, one or more slots 256 (e.g., two shown in FIG. 6) may be formed on at least one of the first insertion surface 252 and the second insertion surface (not shown). The slots 256 may be formed at a anterior portion of the insertion 250 and may extend laterally along the anterior surface 206B. The slots 256 may be tapered from a bottom (or inferior) end to an open upper (or superior) end thereof. The shell 210 may have one or more guides 236 (e.g., two shown in FIG. 6) formed corresponding to the one or more slots 256, respectively. The guides 236 may be tapered to fit the tapered slots 256 of the insertion 250. The shell 210 and the insertion 250 may be conjoined by aligning an end of the guide 236 with an end of the slot 256 and then pushing the insertion 250 in a direction shown as arrow A into the space surround by the shell 210 (or pushing the shell 210 toward the insertion 250 in the direction opposite to arrow A). The tapered guides 236 and the slots 256 may form a dovetail-like joint that holds the shell 210 and the insertion 250 together.

The cage structure(s) described herein, including cage structure 200 (or 100) may include additional features, constructed according to the principles of the disclosure. For instance, the cage structures described herein may include one or more anchoring ears that may be integrally formed with the cage structures.

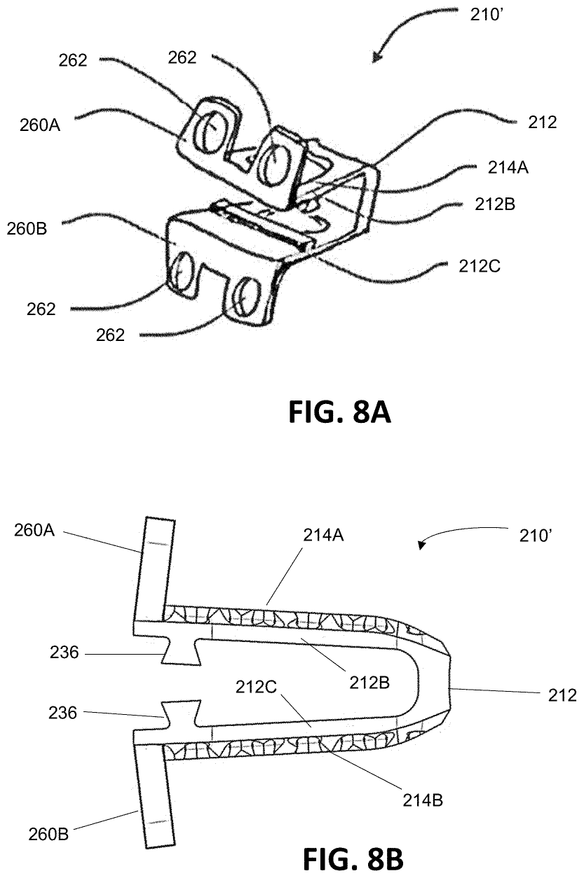

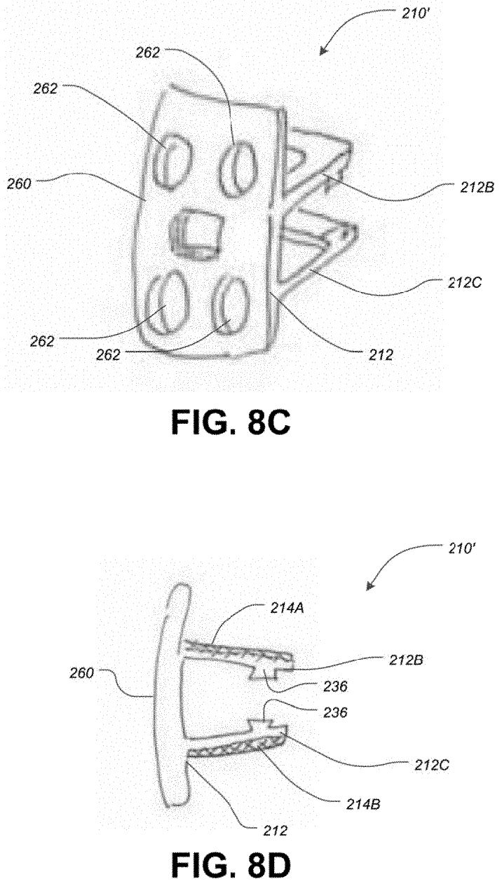

FIGS. 8A and 8B illustrate a further embodiment of the cage structure 200 (or 100). The cage structure 200 (or 100) may include one or more anchoring ears that may be integrally formed with the shell 200 (shown in FIG. 5B), or the main body 110 (shown in FIG. 4B), or one or more of the plates 150A, 150B (shown in FIG. 4B).

Referring to FIGS. 8A and 8B, the cage structure 200 (or 100) may include one or more bone anchoring ears 260A, 260B. As seen in FIGS. 8A, 8B, the cage structure may include the shell 210′, which includes the bone anchoring ears 260A, 260B. The bone anchoring ears 260A, 260B may include one or more screw holes 262. The bone anchoring ears 260A, 260B may be integrally formed with the main body 212 of the shell 210′. For example, the wing portions 212B, 212C of the main body 212 may have portions extending beyond the surface layers 214A, 214B, respectively. The extended portions of the wing portions 212B, 212C may be drilled to form the screw holes 262 and may then be bent away from each other to form the ears 260A, 260B, respectively. Alternatively, the ears 260A, 260B may be produced independently and then attached to edges of the wings 212B, 212C of the main body 210, respectively. Alternatively, the ears 260A, 260B may be formed with the wing portions 212B, 212C, including holes therein, and bent, as understood by those skilled in the art.

The cage structure 200 may be modified to include screw holes without adding the bone anchoring ears 260A, 260B shown in FIGS. 8A and 8B.

FIGS. 8C and 8D illustrate a further example of a cage structure 200 that is constructed according to the principles of the disclosure.

Referring to FIGS. 8C and 8D, the cage structure 200 may include a shell 210′ having an anterior coronal face 260 and one or more screw holes (e.g., four) 262. The face 260 may be integrally formed with the main body 212 of the shell 210′. As seen in FIG. 8D, the wing portions 212B, 212C of the main body 212 may have the surface layers 214A, 214B, respectively, which may be integrally formed with the main body 212 or attached as plates (such as, e.g., plates 150A, 150B, shown in FIGS. 3A-4A. The wing portions 212B, 212C may include the tapered guides 236 to receive and guide an insertion 250.

The cage shell 210′ may be implanted in a patient using a process similar to that described for the interbody device 410 or interbody system 400 described in U.S. patent application Ser. No. 15/244,868, filed Aug. 23, 2016 and entitled “Modular Plate and Cage Elements and Related Methods,” the entirety of which is incorporated herein by reference, with references to FIGS. 18A-18C of that application.

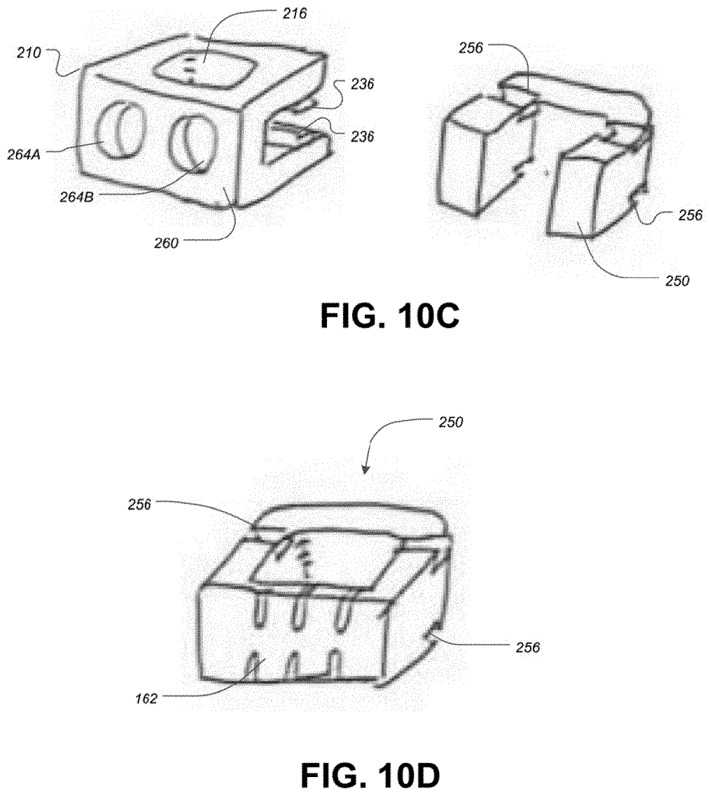

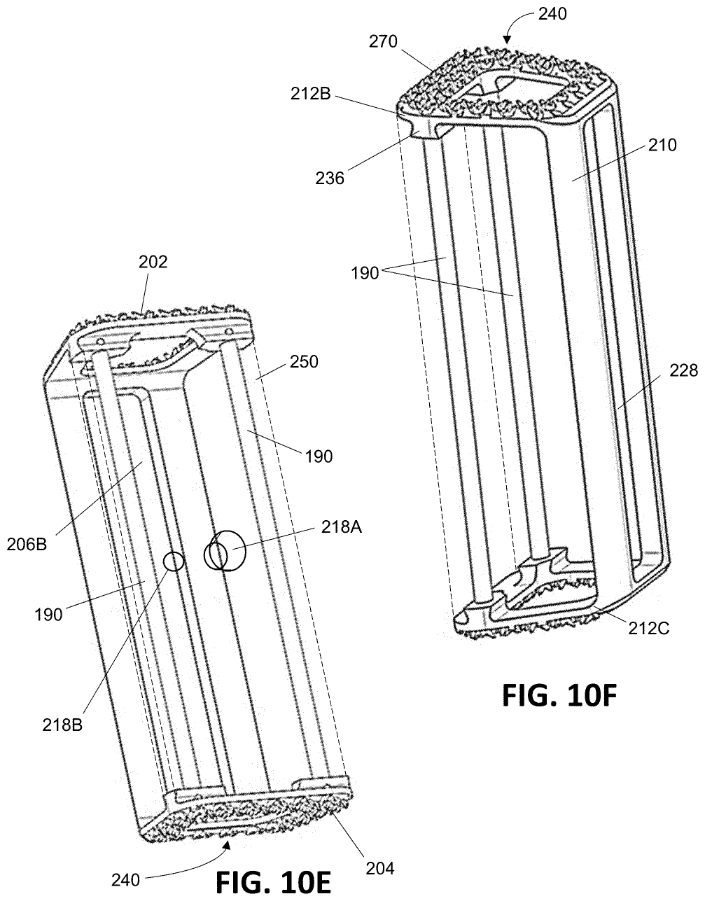

FIGS. 10A and 10B illustrate a cage structure 200 having a modified insertion 250, which is constructed according to the principles of the disclosure. The modified insertion 250 may include one or more screw holes 264A, 264B, which may extend from the anterior surface 206B to the inner surface 216. As seen in FIG. 10B, one or more screws 266A, 266B may be inserted into the corresponding screw holes 266A, 266B. The screw hole 264A may be slanted to direct the screw 266A upwardly, and the screw hole 264B may be slanted to direct the screw 266B downwardly.

FIG. 10C illustrates another example of a cage structure 200′ that is constructed according to the principles of the disclosure. As seen, the cage structure 200′ may comprise the shell 210 and/or the insertion 250, wherein the insertion 250 may include superior and/or inferior slots 256 that align with and engage corresponding one or more guides 236 on the shell 210. The insertion 250 may have an open arrangement (shown in FIG. 10C) or a closed arrangement (shown in FIG. 10D).

FIG. 10D illustrates an example of an insertion 250 have a closed arrangement. As seen in FIG. 10D, at least one of the walls may be formed by a thin wall membrane 162, which is illustrated and described in U.S. patent application Ser. No. 15/244,868, filed Aug. 23, 2016 and entitled “Modular Plate and Cage Elements and Related Methods,” the entirety of which is incorporated herein by reference.

FIGS. 10E and 10F illustrate perspective anterior and lateral views, respectively, of another example of a cage structure constructed according to the principles of the disclosure. The cage structure seen in FIGS. 10E and 10F may be used in corpectomy applications. The cage structure includes the shell 210 and insertion 250, which when assembled may have a height that may range from, for example, about 4 mm to about 200 mm. Other heights are contemplated herein, including less than 4 mm or greater than 200 mm.

As seen in FIGS. 10E and 10F, the cage structure may include one or more holes (or openings), such as, for example, hole 218A and hole or recessed portion 218B. Alternatively (or additionally), the cage structure may include fastening holes (not shown) that may be configured to receive one or more bone fasteners (e.g., bone screws 12 shown in FIG. 2) to secure the cage structure to vertebrae. In this regard, the fastening holes (not shown) may be angled so as to guide the bone fasteners toward and into the vertebrae. FIG. 2 shows an example of fastening holes formed in an implantable device and angled so as to guide the bone screws 12 toward and into adjacent vertebrae 4.

The holes 218A, 218B may extend inwardly from the anterior surface 206B to engage, for example, the implant tool 400 (shown in FIG. 4B) or the like. For example, similar to the holes 108A, 108B of the cage structure 100, the holes 218A, 218B may be constructed to engage the threaded end 432 of the inner shaft and an orientation guide, respectively, of the implant tool 400, shown in FIG. 4B.

The cage structure may include one or more openings 240, which may extend from the first surface 202 to the second surface 204. The opening 240 may be a graft chamber, as discussed above. As seen in FIGS. 10E and 10F, the opening 240 may be formed at, for example, a center portion of the cage structure. The opening 240 may be laterally surrounded and defined by inner wall surfaces of the insertion 250 and shell 210. The shell 210 may include an opening 228. The shell 210 may be secured to the insertion 250 via one or more fasteners (e.g., two) 190. For instance, once the insertion 250 is inserted between the wing portions 212B, 212C along guides 236 and located in its final assembly position upper (shown in FIGS. 10E, 10F), the fasteners 190 may be inserted at a surface of the wing portion 212B (or 212C) and longitudinally through the insertion 250 to and through the other wing portion 212C (or 212B), whereby the fastener 190 will secure the shell 210 to the insertion 250.

The first and second surfaces 202, 204 may have a surface pattern 270, which may be configured to directly contact a surface of the adjacent vertebra during implantation. The surface pattern 270 may establish and promote bone growth and resist movement (e.g., departure, slippage, or the like), as described above.

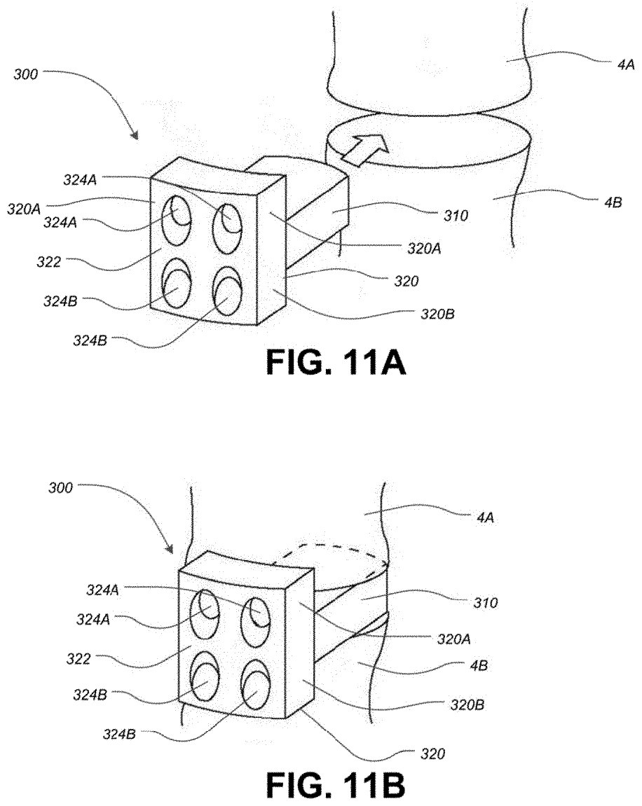

FIGS. 11A and 11B illustrate another example of a cage structure 300, which is constructed according to the principles of the disclosure. The cage structure 300 may be constructed with an insertion portion 310 and a mounting plate 320. The insertion portion 310 may be any cage that is inserted between adjacent vertebrae 4A, 4B. For example, the insertion portion 310 may be the cage structure 200 shown in FIG. 5A or the cage structure 100 shown in FIGS. 3A-4A. The mounting plate 320 may have a first main surface 322 and a second main surface (not shown) located opposite to the first main surface 322. The insertion portion 310 may be connected to a center portion of the second main surface (not shown), which divides the mounting plate 320 into an upper portion 320A and a lower portion 320B.

The mounting plate 320 may include a plurality of screw holes which extend from the first main surface 322 to the second main surface (not shown). For example, one or more screw holes 324A (two shown) may be formed at the upper portion 320A, and one or more screw holes 324B (two shown) may be formed at the lower portion 320B. The screw holes 324A formed at the upper portion 320A may be slanted upwardly to direct bone screws (not shown) inserted thereto further up from a bottom of the vertebrae 4A. The screw holes 324B formed at the lower portion 320B may be slanted downwardly to direct bone screws (not shown) inserted thereto further down from a top of the vertebrae 4B. The insertion portion 310 and the mounting plate 320 may be integrally formed, or, alternatively, produced independently from each other and assembled together.

While the disclosure has been described in terms of exemplary embodiments, those skilled in the art will recognize that the disclosure can be practiced with modifications in the spirit and scope of the appended claim, drawings and attachment. The examples provided herein are merely illustrative and are not meant to be an exhaustive list of all possible designs, embodiments, applications or modifications of the disclosure

What is claimed is:

1. An intervertebral cage structure comprising:
a main body comprising a first upper surface and a second lower surface located opposite to the first surface;
a generally C-shaped outer shell body comprising a first generally planar upper plate, a second generally planar lower plate and a bridge portion connecting the first generally planar upper plate to the second generally planar lower plate, an inner surface of the first generally planar upper plate disposed on the first upper surface of the main body and an inner surface of the second generally planar lower plate disposed on the second lower surface of the main body; and
an opening formed at a center portion of the intervertebral cage structure and extending from an opening in the first generally planar upper plate to an opening in the second generally planar lower plate via the main body,
wherein at least one of the first and second generally planar plates comprise a surface pattern on an outwardly facing surface of the at least one of the first and second generally planar plates comprising a first plurality of protrusions extending outward from the outwardly facing surface of the at least one of the first and second generally planar plates, each of the first plurality of protrusions having at least a first undercut portion and a second undercut portion, the second undercut portion being non-parallel to the first undercut portion.

2. The intervertebral cage structure of claim 1, wherein the main body comprises Polyether Ether Ketone (PEEK) and the generally C-shaped outer shell body comprises titanium.

3. The intervertebral cage structure of claim 1, wherein the main body further comprises a plurality of lateral surfaces extending between the first upper and second lower surfaces; and
one or more holes extending from one of the plurality of lateral surfaces towards the opening.

4. The intervertebral cage structure of claim 3, wherein the main body further comprises an inner surface surrounding the opening, the inner surface comprising a bulged portion surrounding a portion of the one or more holes.

5. The intervertebral cage structure of claim 1, further comprising:
a pin hole extending from the first generally planar upper plate to the main body; and
a pin that inserts into the pin hole,
wherein the intervertebral cage is configured for a corpectomy application.

6. The intervertebral cage structure of claim 1, wherein the main body further comprises at least one slot, and wherein the inner surface of the first plate comprises at least one tab that inserts into the at least one slot of the main body to secure the first plate to the main body.

7. An intervertebral cage structure comprising:
a main body having a first surface and a second surface located opposite to the first surface;
a clamshell shaped outer shell body comprising a first plate having an outer surface and an inner surface, the inner surface of the first plate engaging with and covering the first surface of the main body; and
an opening formed in the intervertebral cage structure and extending from the first surface to the second surface located opposite the first surface of the main body,
wherein the outer surface of the first plate has an outwardly extending surface pattern comprising a plurality of symmetrically distributed protrusions, each of the symmetrically distributed protrusions having at least a first undercut portion and a second undercut portion, the second undercut portion being non-parallel to the first undercut portion, at least a portion of the symmetrically distributed protrusions having a pocket formed in a superior surface thereof.

8. The intervertebral cage structure of claim 7, wherein the main body comprises PEEK and the first plate comprise titanium or a titanium alloy.

9. The intervertebral cage structure of claim 7, further comprising a second plate, the first plate and the second plate forming the outer shell main body, wherein the outer shell main body is configured to receive and substantially encapsulate the main body.

10. The intervertebral cage structure of claim 9, wherein the outer shell main body comprises a clam shape that includes said first plate and the second plate connected by a bridge portion.

11. The intervertebral cage structure of claim 7, wherein the main body comprises at least one of the members consisting of the group of a metal, PEEK, silicon and allograft.

12. The intervertebral cage structure of claim 7, wherein the main body further comprises:
a plurality of lateral surfaces extending between the first and second surfaces; and
one or more holes extending from one of the plurality of lateral surfaces towards the opening.

13. The intervertebral cage structure of claim 12, wherein the main body further comprises an inner surface surrounding the opening, the inner surface comprising a bulged portion surrounding a portion of the one or more holes.

14. The intervertebral cage structure of claim 7, further comprising:
a pin hole extending from the plate to the main body; and
a pin that inserts into the pin hole.

15. The intervertebral cage structure of claim 7, wherein the main body further comprises one or more slots, and
wherein the inner surface of the first plate comprises one or more tabs that insert into the one or more slots of the main body to secure the first plate to the main body.

16. The intervertebral cage structure of claim 7, wherein each of the symmetrically distributed protrusions further comprise a plurality of outwardly extending prongs separated by the pocket.

17. An intervertebral cage structure comprising:
a main body having a surface;
a clamshell shaped plate disposed substantially around the surface of the main body; and
an opening formed in the intervertebral cage structure and extending from the surface and through the main body,
wherein the intervertebral cage structure has a surface pattern that comprises a plurality of protrusions, each of the plurality of protrusions including a centrally positioned pocket, each of the plurality of protrusions further having a first undercut portion having a first orientation and a second undercut portion having a second orientation, the first orientation being non-parallel to the second orientation.

18. The intervertebral cage structure of claim 17, wherein the plate comprises titanium and the main body comprises PEEK.

19. The intervertebral cage structure of claim 17, wherein each centrally positioned pocket includes a pocket surface, the plurality of protrusions being separated by a plurality of depressions positioned therebetween, the depressions including a depression surface, wherein the pocket surfaces are proud of the depression surfaces.

20. The intervertebral cage structure of claim 17, wherein the plate comprises a metallic material and the main body comprises a material containing silicon.
